(19) United States
(12) Patent Application Publication
(10) Pub. No.: US 2025/0288641 A1
(43) Pub. Date: Sep. 18, 2025

(54) MICRO-DYSTROPHIN FOR HEART PROTECTION

(71) Applicant: The Curators of the University of Missouri, Columbia, MO (US)

(72) Inventors: Dongsheng Duan, Columbia, MO (US); Xiufang Pan, Columbia, MO (US); Yongping Yue, Coumbia, MO (US)

(21) Appl. No.: 18/860,189

(22) PCT Filed: Apr. 27, 2023
(86) PCT No.: PCT/US2023/020202

§ 371 Date: Oct. 25, 2024

Related U.S. Application Data
(60) Provisional application No. 63/335,414, filed on Apr. 27, 2022.

(51) Int. Cl. A61K 38/17; A61K 48/00; A61P 21/00; C07K 14/47; C12N 15/86

(52) U.S. Cl. CPC A61K 38/1709; A61K 48/0058; A61P 21/00; C07K 14/4708; C12N 15/86; C12N 2750/14143; C12N 2830/008

(57) ABSTRACT

Disclosed are compositions and methods for treating Duchenne muscular dystrophy. The compositions include a micro-dystrophin having a dystrophin N-terminal domain, a dystrophin R16 domain, a dystrophin R17 domain, a dystrophin R18 domain, a dystrophin R19 domain, and a dystrophin CR domain. The compositions are particularly useful for treating Duchenne muscular dystrophy and for treating cardiac arrhythmia in subjects having or suspected of having Duchenne muscular dystrophy.

### CROSS-REFERENCE TO RELATED APPLICATIONS

This application claims priority to U.S. Provisional Patent Application No. 63/335,414, filed on Apr. 27, 2022, which is incorporated by reference in its entirety.

### STATEMENT OF GOVERNMENT SUPPORT

The invention was made with government support under W81XWH-14-1-0302 awarded by the Medical Research and Development Command, and R01 HL091883, and R01 NS090634 awarded by the National Institutes of Health. The government has certain rights in the invention.

### BACKGROUND OF THE DISCLOSURE

The present disclosure relates generally to medicine. More particularly, the present disclosure is directed to compositions and methods for treating Duchenne muscular dystrophy. The compositions and methods are particularly suitable for treating cardiac arrhythmia in subjects having or suspected of having Duchenne muscular dystrophy. Also disclosed are compositions including a micro-dystrophin having a dystrophin N-terminal domain, a dystrophin R16 domain, a dystrophin R17 domain, a dystrophin R18 domain, a dystrophin R19 domain, and a dystrophin CR domain and viral vectors that express a micro-dystrophin having a dystrophin N-terminal domain, a dystrophin R16 domain, a dystrophin R17 domain, a dystrophin R18 domain, a dystrophin R19 domain, and a dystrophin CR domain.

Dystrophin deficiency leads to Duchenne muscular dystrophy (DMD). Patients often die from skeletal muscle failure-associated respiratory disease and/or dilated cardiomyopathy. While much has been learned on how dystrophin protects skeletal muscle, little is known on how dystrophin effects cardiac protection. Despite the similarity between skeletal muscle and cardiac muscle, these two tissues have major structural and functional differences. It is thus not surprising that dystrophin domains important for skeletal muscle protection may not necessarily be important for heart protection and vice versa.

Cardiac arrhythmias significantly contribute to mortality in Duchenne muscular dystrophy (DMD), a disease caused by dystrophin deficiency. A major source of arrhythmias in patients with DMD is impaired ventricular impulse conduction, which predisposes for ventricular asynchrony, decreased cardiac output, and the development of reentrant mechanisms. Using the mdx mouse model for DMD, lack of dystrophin was shown to cause considerable Na current loss in Purkinje fibers, cardiomyocytes specialized for electrical impulse conduction.

Several domains important for skeletal muscle protection (such the R16/17 nNOS-binding domain) have been discovered. However, a dystrophin heart protection domain has never been identified. The existence of a heart protection domain has been suspected based on genotype-phenotype analysis of patient data. Yet, the nature of a dystrophin heart protection domain has been elusive. Accordingly, there exists an ongoing need for developing new compositions and methods for treating Duchenne muscular dystrophy and, in particular, for treating cardiomyopathies in subjects having or suspected of having Duchenne muscular dystrophy.

### BRIEF DESCRIPTION OF THE DISCLOSURE

The present disclosure relates generally to compositions and methods for treating Duchenne muscular dystrophy. The compositions and methods are particularly useful for treating cardiac arrhythmias in subjects having or suspected of having Duchenne muscular dystrophy.

In one aspect, the present disclosure is directed to a composition for treating Duchenne muscular dystrophy comprising a virus comprising a nucleic acid sequence encoding a micro-dystrophin that comprises a dystrophin N-terminal domain, a dystrophin R16 domain, a dystrophin R17 domain, a dystrophin R18 domain, a dystrophin R19 domain, and a dystrophin CR domain.

In one aspect, the present disclosure is directed to a method of treating Duchenne muscular dystrophy in a subject having or suspected of having Duchenne muscular dystrophy, the method comprising, administering to the subject a micro-dystrophin comprising a dystrophin N-terminal domain, a dystrophin R16 domain, a dystrophin R17 domain, a dystrophin R18 domain, a dystrophin R19 domain, and a dystrophin CR domain.

In one aspect, the present disclosure is directed to a method of treating cardiac arrhythmia in a subject having or suspected of having Duchenne muscular dystrophy, the method comprising, administering to the subject a micro-dystrophin comprising a dystrophin N-terminal domain, a dystrophin R16 domain, a dystrophin R17 domain, a dystrophin R18 domain, a dystrophin R19 domain, and a dystrophin CR domain.

### DESCRIPTION OF THE FIGURES

FIGS. 1A-1J depict the full rescue of impaired sodium currents in dystrophic Purkinje fibers by microdystrophin therapy. FIG. 1A depicts the full-length dystrophin and micro-dystrophin structure.

FIG. 1B depicts representative dystrophin immunofluorescence staining photomicrographs in the heart of mdx-Cx40_{eGFP/+} mice at 12 weeks after AAV micro-dystrophin injection. Dystrophin R17 was detected with Manex 44A antibody (1:500; gift from Dr. Glenn Morris at the Robert Jones and Agnes Hunt Orthopedic Hospital, Oswestry, UK). Dystrophin C-terminal domain (CT) was recognized by Dys-2 antibody (1:20; Novocastra, Newcastle, UK).

FIG. 1C depicts representative dystrophin (R17)/laminin (1:200; Sigma, St. Louis, MO) double immunostaining photomicrographs illustrating saturated micro-dystrophin expression in the heart of injected mice.

FIG. 1D depicts quantification of dystrophin positive cardiomyocytes. For each group, hearts from 8 mice (4 randomly chosen sections per heart) were used.

FIG. 1E depicts micro-dystrophin western blot analysis using the Manex 44A antibody (against dystrophin R17, 1:100); Filled triangle, micro-dystrophin (132 kD): Open triangle, alpha-tubulin (50 kD).

FIG. 1F depicts typical original Ca current (ICa) traces of a wt-Cx40_{eGFP/+} cell, elicited by the pulse protocol displayed on top. The respective current density-voltage relationship at the bottom shows the presence of considerable T-type Ca current, typical for Purkinje fibers but not ventricular cardiomyocytes. A series of control experiments revealed that 25 out of 26 tested wt-Cx40_{eGFP/+} cells, and all 18 tested mdx-Cx40_{eGFP/+} cells, had a T-type Ca current amplitude of at least 33% when compared with the respective cells' L-type Ca current amplitudes, confirming the eGFP fluorescence signal as robust indicator of Purkinje fiber identity.

FIG. 1G depicts typical whole cell Na currents recorded from a wild-type (wt), and untreated mdx, and a μDys-treated mdx Purkinje fiber at room temperature (pulse protocol, inset). The bath solution contained (in mM): 5 NaCl, 135 NMDG, 2.5 KCl, 1 CaCl₂, 1 MgCl₂, 10 HEPES; pH=7.4, adjusted with HCl. Pipette solution: 5 NaCl, 110 CsF, 10 EGTA, 10 HEPES; pH=7.3, adjusted with CsOH.

FIG. 1H depicts current density-voltage relationships (left), and current density values at −38 mV (right) (n=58 cells from 8 wt hearts; n=37 cells from 6 mdx hearts; n=20 cells from 6 mdx μDys hearts).

FIG. 1I depicts representative peak amplitude-normalized Na current decay (left), and comparison of decay half-times (right) at −38 mV. Decay half-time represents the time period between the current peak and the time points at which the current had decaved to 50% (see arrows).

FIG. 1J depicts comparison of cell capacitance values for cell size estimation. Data are given as means±SE. Statistical comparisons were performed using a nested analysis respecting the hierarchical data structure (measurements of n cells from m animals) (* p<0.05, ** p<0.01, *** p<0.001; p>0.05, ns, not significant).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure.

Unless defined otherwise, all technical and scientific terms used herein have the same meaning as commonly understood by one of skill in the art to which the invention pertains. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.” The term “about” means up to ±10%.

As used herein, “susceptible” and “at risk” refer to having little resistance to a certain disease, disorder or condition, including being genetically predisposed, having a family history of, and/or having symptoms of the disease, disorder or condition.

As used herein, a subject (also used interchangeably with “patient”) in need thereof, as it relates to the therapeutic uses herein, is one identified to require or desire medical intervention. Because some of the method embodiments of the present disclosure are directed to specific subsets or subclasses of identified subject (that is, the subset or subclass of subject “in need” of assistance in addressing one or more specific conditions noted herein), not all subjects will fall within the subset or subclass of subjects in need of treatment described herein. An effective amount is that amount of an agent necessary to inhibit the pathological diseases and disorders herein described. When at least one additional therapeutic agent is administered to a subject, such agent may be administered sequentially, concurrently, or simultaneously, in order to obtain the benefits of the agents. The term subject includes vertebrate animals, and preferably is a human patient.

In one aspect, the present disclosure is directed to a composition for treating Duchenne muscular dystrophy in a subject having or suspected of having Duchenne muscular dystrophy. The composition includes a micro-dystrophin that includes a dystrophin N-terminal domain, a dystrophin repeat 16 (R16) domain, a dystrophin repeat 17 (R17) domain, a dystrophin repeat 18 (R18) domain, a dystrophin repeat 19 (R19) domain, and a dystrophin cysteine rich (CR) domain. Dystrophin repeat 16-19 domains (referred to herein as “dystrophin R16-19 domains” and “R16-19”) are spectrin-like repeats 16 to 19 of the dystrophin protein. Dystrophin CR domain refers to dystrophin cysteine-rich domains.

The micro-dystrophin can further include a dystrophin hinge 1 (H1) domain. The micro-dystrophin can further include a dystrophin hinge 4 (H4) domain.

In one embodiment, the composition is a nucleic acid encoding the micro-dystrophin. In another aspect, the composition is a micro-dystrophin protein. The micro-dystrophin protein can be recombinantly produced using well-known recombinant protein expression methods and/or chemically synthesized micro-dystrophin protein.

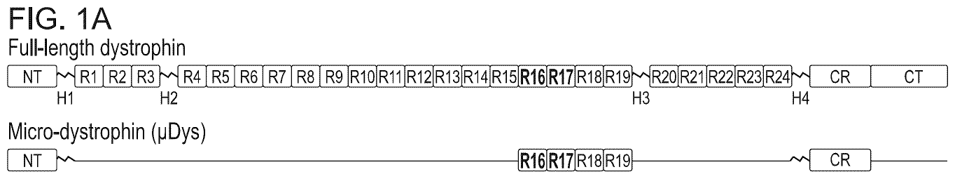

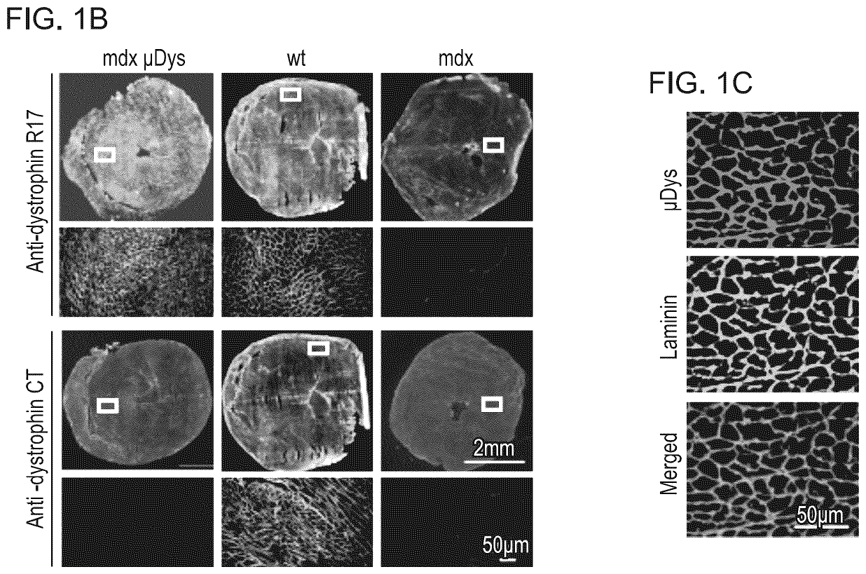

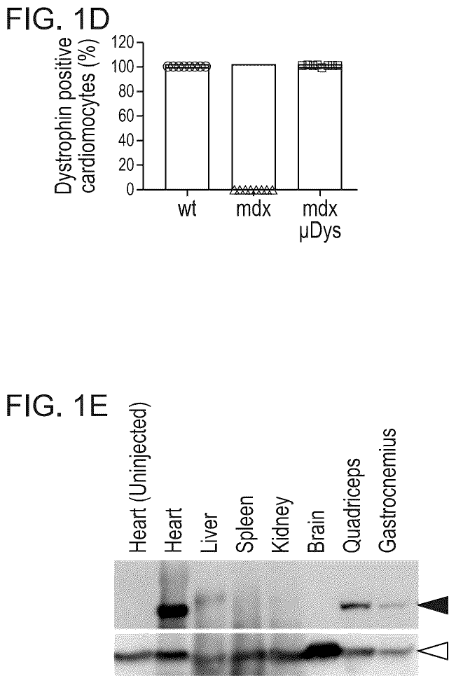

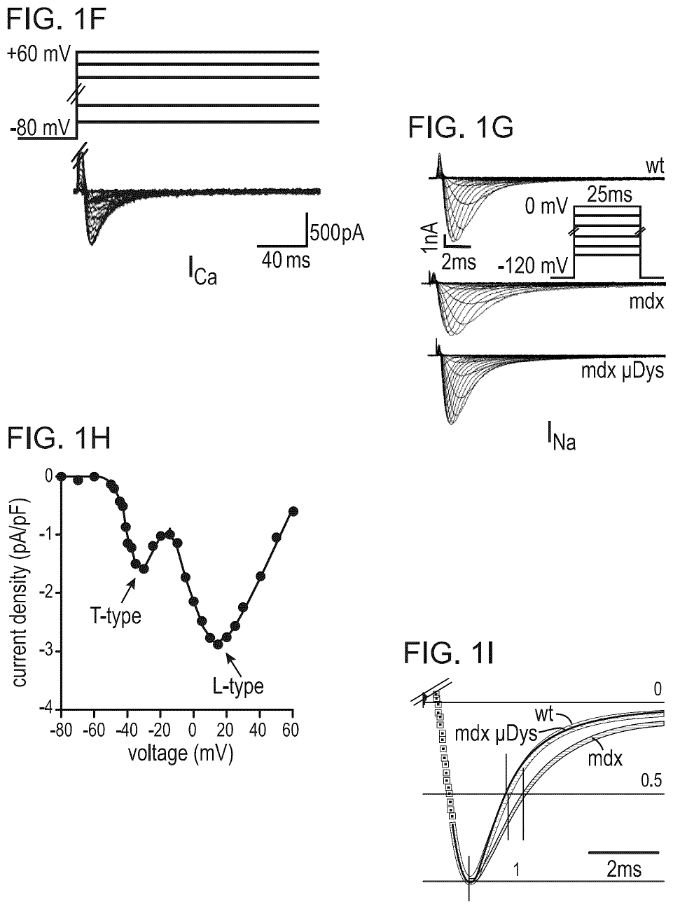

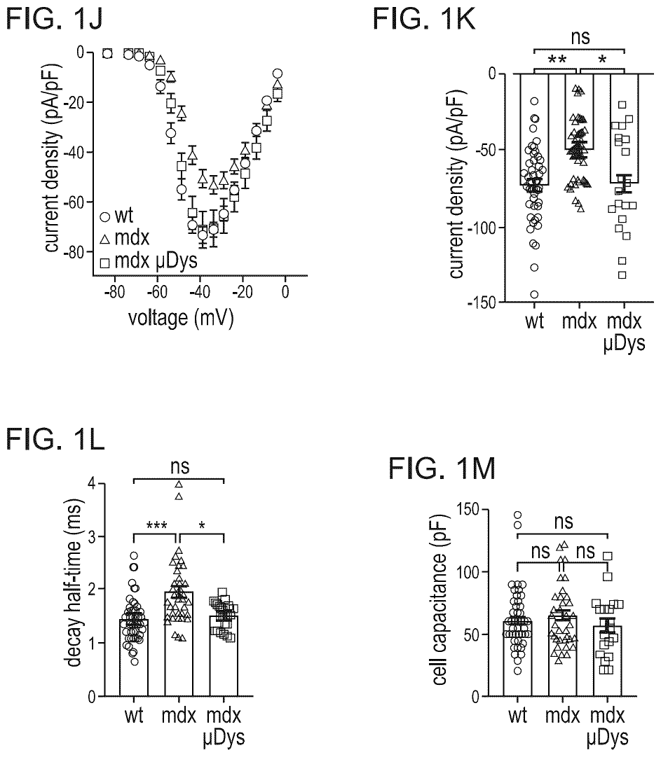

FIG. 1B illustrates the protein domain structure of an exemplary embodiment of a micro-dystrophin that includes a dystrophin N-terminal domain (NT), a hinge 1 (H1) domain, a dystrophin R16 domain, a dystrophin R17 domain, a dystrophin R18 domain, a dystrophin R19 domain, a hinge 4 (H4) domain, and a dystrophin CR domain as compared to a full-length dystrophin protein domain structure having a dystrophin N-terminal (NT), a hinge 1 (H1) domain, R1-R3 domains, a hinge 2 (H2) domain, R4-R19 domains, a hinge 3 (H3) domain, R20-R24 domains, a hinge 4 (H4) domain, a CR domain (CR), and a C-terminal domain (CT).

The composition can be in the form of a polypeptide of a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can also be in the form of a nucleic acid sequence that encodes a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can be in the form of a viral vector including a nucleic acid sequence encoding a micro-dystrophin that includes a dystrophin N-terminal domain, a dystrophin R16 domain, a dystrophin R17 domain, a dystrophin R18 domain, a dystrophin R19 domain, and a dystrophin CR domain.

The nucleic acid can be a vector. Suitable vectors include viral vectors and non-viral vectors. Suitable viral vectors include adeno-associated viral vectors. Particularly suitable adeno-associated viral vectors include an adeno-associated-virus serotype-1 (AVV-1) viral vector, an adeno-associated-virus serotype-2 (AVV-2) viral vector, adeno-associated-virus serotype-5 (AVV-5) viral vector, adeno-associated-virus serotype-6 (AVV-6) viral vector, adeno-associated-virus serotype-8 (AVV-8) viral vector, adeno-associated-virus serotype-9 (AVV-9) viral vector, adeno-associated-virus serotype-rh74 (AVV-rh74) viral vector, adeno-associated-virus-218 (AVV-2i8) viral vector, adeno-associated-virus-B1 (AVV-B1) viral vector, adeno-associated-virus-CAM130 (AVV-CAM130) viral vector, adeno-associated-virus-M41 (AVV-M41) viral vector, adeno-associated-virus MTP (AAV587MTP and AAV588MTP) viral vector, adeno-associated-virus NP22 (AAV-NP22) viral vector, adeno-associated-virus NP66 (AAV-NP66) viral vector, adeno-associated-virus MYO (AAVMYO) viral vector, an adeno-associated-virus tyrosine mutant viral vector, and an ancestral adeno-associated-virus (ancAVV) viral vector.

The nucleic acid encoding the micro-dystrophins of the present disclosure can further include a promoter. Particularly suitable promoters include tissue-specific promoters such as cardiac-specific promoters, skeletal muscle promoters, and striated muscle promoters. A particularly suitable cardiac-specific promoter includes an a-myosin heavy chain promoter.

The composition can further include other components such as surfactants, preservatives, and excipients. Surfactants can reduce or prevent surface-induced aggregation of the dystrophin microgenes. Suitable surfactants fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. The use of a preservative in pharmaceutical compositions is well-known to those skilled in the art. For convenience reference is made to Remington: The Science and Practice of Pharmacy, 19th edition, 1995. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

The composition can further include a pharmaceutically acceptable carrier. As understood by those skilled in the art, pharmaceutically acceptable carriers, and, optionally, other therapeutic and/or prophylactic ingredients must be “acceptable” in the sense of being compatible with the other ingredients of the formulation and not be harmful to the recipient thereof. Suitable pharmaceutically acceptable carrier solutions include water, saline, isotonic saline, phosphate buffered saline, Ringer's lactate, and the like. The compositions of the present disclosure can be administered to animals, preferably to mammals, and in particular to humans as therapeutics per se, as mixtures with one another or in the form of pharmaceutical preparations, and which as active constituent contains an effective dose of the active agent, in addition to customary pharmaceutically innocuous excipients and additives.

In one aspect, the present disclosure is directed to a vector including a nucleic acid encoding a micro-dystrophin comprising a dystrophin N-terminal domain, a dystrophin R16 domain, a dystrophin R17 domain, a dystrophin R18 domain, a dystrophin R19 domain, and a dystrophin CR domain.

The vector including a nucleic acid encoding the micro-dystrophin can further include a dystrophin hinge 1 (H1) domain. The vector including a nucleic acid encoding the micro-dystrophin can further include a dystrophin hinge 4 (H4) domain.

The vector can be viral vectors and non-viral vectors. Suitable viral vectors include adeno-associated viral vectors. Suitable adeno-associated viral vectors include an adeno-associated-virus serotype-1 (AVV-1) viral vector, an adeno-associated-virus serotype-2 (AVV-2) viral vector, adeno-associated-virus serotype-5 (AVV-5) viral vector, adeno-associated-virus serotype-6 (AVV-6) viral vector, adeno-associated-virus serotype-8 (AVV-8) viral vector, adeno-associated-virus serotype-9(AVV-9) viral vector, adeno-associated-virus serotype-rh74 (AVV-rh74) viral vector, adeno-associated-virus-218 (AVV-218) viral vector, adeno-associated-virus-B1 (AVV-B1) viral vector, adeno-associated-virus-CAM130 (AVV-CAM130) viral vector, adeno-associated-virus-M41 (AVV-M41) viral vector, adeno-associated-virus MTP (AAV587MTP and AAV588MTP) viral vector, adeno-associated-virus NP22 (AAV-NP22) viral vector, adeno-associated-virus NP66 (AAV-NP66) viral vector, adeno-associated-virus MYO (AAVMYO) viral vector, an adeno-associated-virus tyrosine mutant viral vector, and an ancestral adeno-associated-virus (ancAVV) viral vector.

The vector including the micro-dystrophins of the present disclosure can further include a promoter. Particularly suitable promoters include tissue-specific promoters such as cardiac-specific promoters, skeletal muscle promoters, and striated muscle promoters. A particularly suitable cardiac-specific promoter includes an α-myosin heavy chain promoter.

In one aspect, the present disclosure is directed to a method of treating Duchenne muscular dystrophy in a subject having or suspected of having Duchenne muscular dystrophy, the method includes: administering to the subject a composition including a micro-dystrophin including a dystrophin N-terminal domain, a dystrophin R16 domain, a dystrophin R17 domain, a dystrophin R18 domain, a dystrophin R19 domain, and a dystrophin CR domain.

The micro-dystrophin can further include a dystrophin hinge 1 (H1) domain. The micro-dystrophin can further include a dystrophin hinge 4 (H4) domain.

The composition can be in the form of a polypeptide of a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can also be in the form of a nucleic acid sequence that encodes a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can be in the form of a vector including a nucleic acid sequence encoding a micro-dystrophin that includes a dystrophin N-terminal domain, a dystrophin R16 domain, a dystrophin R17 domain, a dystrophin R18 domain, a dystrophin R19 domain, and a dystrophin CR domain.

The micro-dystrophin is suitably administered to the subject in suitable vectors. Suitable vectors include adeno-associated virus (AAV). Suitable adeno-associated viral vectors include an adeno-associated-virus serotype-1 (AVV-1) viral vector, an adeno-associated-virus serotype-2 (AVV-2) viral vector, adeno-associated-virus serotype-5 (AVV-5) viral vector, adeno-associated-virus serotype-6 (AVV-6) viral vector, adeno-associated-virus serotype-8 (AVV-8) viral vector, adeno-associated-virus serotype-9 (AVV-9) viral vector, adeno-associated-virus serotype-rh74 (AVV-rh74) viral vector, adeno-associated-virus-218 (AVV-2i8) viral vector, adeno-associated-virus-B1 (AVV-B1) viral vector, adeno-associated-virus-CAM130(AVV-CAM130) viral vector, adeno-associated-virus-M41 (AVV-M41) viral vector, adeno-associated-virus MTP (AAV587MTP and AAV588MTP) viral vector, adeno-associated-virus NP22 (AAV-NP22) viral vector, adeno-associated-virus NP66 (AAV-NP66) viral vector, adeno-associated-virus MYO (AAVMYO) viral vector, an adeno-associated-virus tyrosine mutant viral vector, and an ancestral adeno-associated-virus (ancAVV) viral vector. Particularly suitable AAV to deliver the micro-dystrophin include adeno-associated virus serotype 2 (AAV2) and adeno-associated virus serotype 9 (AAV9).

Nucleic acid sequences encoding a micro-dystrophin that includes a dystrophin N-terminal domain, a dystrophin R16 domain, a dystrophin R17 domain, a dystrophin R18 domain, a dystrophin R19 domain, and a dystrophin CR domain are operably linked to a muscle-specific promoter for expression in muscle tissue including skeletal and cardiac muscle. Nucleic acid sequences encoding a micro-dystrophin that includes a dystrophin N-terminal domain, a dystrophin R16 domain, a dystrophin R17 domain, a dystrophin R18 domain, a dystrophin R19 domain, and a dystrophin CR domain are operably linked to a cardiac-specific promoter for expression in cardiac tissue. A particularly suitable cardiac-specific promoter is an a-myosin heavy chain promoter.

The composition can further include other components such as surfactants, preservatives, and excipients. Surfactants can reduce or prevent surface-induced aggregation of the dystrophin microgenes. Suitable surfactants fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. The use of a preservative in pharmaceutical compositions is well-known to those skilled in the art. For convenience reference is made to Remington: The Science and Practice of Pharmacy, 19th edition, 1995. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

The composition can further include a pharmaceutically acceptable carrier. As understood by those skilled in the art, pharmaceutically acceptable carriers, and, optionally, other therapeutic and/or prophylactic ingredients must be “acceptable” in the sense of being compatible with the other ingredients of the formulation and not be harmful to the recipient thereof. Suitable pharmaceutically acceptable carrier solutions include water, saline, isotonic saline, phosphate buffered saline, Ringer's lactate, and the like. The compositions of the present disclosure can be administered to animals, preferably to mammals, and in particular to humans as therapeutics per se, as mixtures with one another or in the form of pharmaceutical preparations, and which as active constituent contains an effective dose of the active agent, in addition to customary pharmaceutically innocuous excipients and additives.

In one aspect, the present disclosure is directed to a method of treating cardiac arrhythmia in a subject having or suspected of having Duchenne muscular dystrophy. The method includes administering to the subject a composition including a micro-dystrophin that includes a dystrophin N-terminal domain, a dystrophin R16 domain, a dystrophin R17 domain, a dystrophin R18 domain, a dystrophin R19 domain, and a dystrophin CR domain.

The micro-dystrophin can further include a dystrophin hinge 1 (H1) domain. The micro-dystrophin can further include a dystrophin hinge 4 (H4) domain.

The composition can be in the form of a polypeptide of a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can also be in the form of a nucleic acid sequence that encodes a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can be in the form of a vector including a nucleic acid sequence encoding a micro-dystrophin that includes a dystrophin N-terminal domain, a dystrophin R16 domain, a dystrophin R17 domain, a dystrophin R18 domain, a dystrophin R19 domain, and a dystrophin CR domain.

The micro-dystrophin is suitably administered to the subject in suitable vectors. Suitable vectors include adeno-associated virus (AAV). Suitable adeno-associated viral vectors include an adeno-associated-virus serotype-1 (AVV-1) viral vector, an adeno-associated-virus serotype-2 (AVV-2) viral vector, adeno-associated-virus serotype-5 (AVV-5) viral vector, adeno-associated-virus serotype-6 (AVV-6) viral vector, adeno-associated-virus serotype-8 (AVV-8) viral vector, adeno-associated-virus serotype-9 (AVV-9) viral vector, adeno-associated-virus serotype-rh74 (AVV-rh74) viral vector, adeno-associated-virus-218 (AVV-218) viral vector, adeno-associated-virus-B1 (AVV-B1) viral vector, adeno-associated-virus-CAM130 (AVV-CAM130) viral vector, adeno-associated-virus-M41 (AVV-M41) viral vector, adeno-associated-virus MTP (AAV587MTP and AAV588MTP) viral vector, adeno-associated-virus NP22 (AAV-NP22) viral vector, adeno-associated-virus NP66 (AAV-NP66) viral vector, adeno-associated-virus MYO (AAVMYO) viral vector, an adeno-associated-virus tyrosine mutant viral vector, and an ancestral adeno-associated-virus (ancAVV) viral vector. Particularly suitable AAV to deliver the micro-dystrophin include adeno-associated virus serotype 2 (AAV2) and adeno-associated virus serotype 9 (AAV9).

Nucleic acid sequences encoding a micro-dystrophin that includes a dystrophin N-terminal domain, a dystrophin R16 domain, a dystrophin R17 domain, a dystrophin R18 domain, a dystrophin R19 domain, and a dystrophin CR domain are operably linked to a muscle-specific promoter for expression in muscle tissue including skeletal and cardiac muscle. Nucleic acid sequences encoding a micro-dystrophin that includes a dystrophin N-terminal domain, a dystrophin R16 domain, a dystrophin R17 domain, a dystrophin R18 domain, a dystrophin R19 domain, and a dystrophin CR domain are operably linked to a cardiac-specific promoter for expression in cardiac tissue. A particularly suitable cardiac-specific promoter is an a-myosin heavy chain promoter.

The composition can further include other components such as surfactants, preservatives, and excipients. Surfactants can reduce or prevent surface-induced aggregation of the dystrophin microgenes. Suitable surfactants fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxy benzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. The use of a preservative in pharmaceutical compositions is well-known to those skilled in the art. For convenience reference is made to Remington: The Science and Practice of Pharmacy, 19th edition, 1995. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

The composition can further include a pharmaceutically acceptable carrier. As understood by those skilled in the art, pharmaceutically acceptable carriers, and, optionally, other therapeutic and/or prophylactic ingredients must be “acceptable” in the sense of being compatible with the other ingredients of the formulation and not be harmful to the recipient thereof. Suitable pharmaceutically acceptable carrier solutions include water, saline, isotonic saline, phosphate buffered saline, Ringer's lactate, and the like. The compositions of the present disclosure can be administered to animals, preferably to mammals, and in particular to humans as therapeutics per se, as mixtures with one another or in the form of pharmaceutical preparations, and which as active constituent contains an effective dose of the active agent, in addition to customary pharmaceutically innocuous excipients and additives.

The method can further include analyzing the subject using electrocardiogram. The methods can further include determining heart hemodynamics.

In one aspect, the present disclosure is directed to a method of providing cardiac protection in a subject having or suspected of having Duchenne muscular dystrophy. The method includes administering to the subject a composition including a micro-dystrophin that includes a dystrophin N-terminal domain, a dystrophin R16 domain, a dystrophin R17 domain, a dystrophin R18 domain, a dystrophin R19 domain, and a dystrophin CR domain. The micro-dystrophin can further include a dystrophin hinge 1 domain and a dystrophin hinge 4 domain.

The micro-dystrophin can further include a dystrophin hinge 1 (H1) domain. The micro-dystrophin can further include a dystrophin hinge 4 (H4) domain.

The composition can be in the form of a polypeptide of a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can also be in the form of a nucleic acid sequence that encodes a micro-dystrophin that includes a dystrophin N-terminal domain, a dystrophin R16 domain, a dystrophin R17 domain, a dystrophin R18 domain, a dystrophin R19 domain, and a dystrophin CR domain. The composition can be in the form of a vector including a nucleic acid sequence encoding a micro-dystrophin that includes a dystrophin N-terminal domain, a dystrophin R16 domain, a dystrophin R17 domain, a dystrophin R18 domain, a dystrophin R19 domain, and a dystrophin CR domain.

The micro-dystrophin is suitably administered to the subject in suitable vectors. Suitable vectors include adeno-associated virus (AAV). Suitable adeno-associated viral vectors include an adeno-associated-virus serotype-1 (AVV-1) viral vector, an adeno-associated-virus serotype-2 (AVV-2) viral vector, adeno-associated-virus serotype-5 (AVV-5) viral vector, adeno-associated-virus serotype-6 (AVV-6) viral vector, adeno-associated-virus serotype-8 (AVV-8) viral vector, adeno-associated-virus serotype-9 (AVV-9) viral vector, adeno-associated-virus serotype-rh74 (AVV-rh74) viral vector, adeno-associated-virus-218 (AVV-218) viral vector, adeno-associated-virus-B1 (AVV-B1) viral vector, adeno-associated-virus-CAM130 (AVV-CAM130) viral vector, adeno-associated-virus-M41 (AVV-M41) viral vector, adeno-associated-virus MTP (AAV587MTP and AAV588MTP) viral vector, adeno-associated-virus NP22 (AAV-NP22) viral vector, adeno-associated-virus NP66 (AAV-NP66) viral vector, adeno-associated-virus MYO (AAVMYO) viral vector, an adeno-associated-virus tyrosine mutant viral vector, and an ancestral adeno-associated-virus (ancAVV) viral vector. Particularly suitable AAV to deliver the micro-dystrophin include adeno-associated virus serotype 2 (AAV2) and adeno-associated virus serotype 9 (AAV9).

Nucleic acid sequences encoding a micro-dystrophin that includes a dystrophin N-terminal domain, a dystrophin R16 domain, a dystrophin R17 domain, a dystrophin R18 domain, a dystrophin R19 domain, and a dystrophin CR domain are operably linked to a muscle-specific promoter for expression in muscle tissue including skeletal and cardiac muscle. Nucleic acid sequences encoding a micro-dystrophin that includes a dystrophin N-terminal domain, a dystrophin R16 domain, a dystrophin R17 domain, a dystrophin R18 domain, a dystrophin R19 domain, and a dystrophin CR domain are operably linked to a cardiac-specific promoter for expression in cardiac tissue. A particularly suitable cardiac-specific promoter is an α-myosin heavy chain promoter.

The composition can further include other components such as surfactants, preservatives, and excipients. Surfactants can reduce or prevent surface-induced aggregation of the dystrophin microgenes. Suitable surfactants fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. The use of a preservative in pharmaceutical compositions is well-known to those skilled in the art. For convenience reference is made to Remington: The Science and Practice of Pharmacy, 19th edition, 1995. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

The composition can further include a pharmaceutically acceptable carrier. As understood by those skilled in the art, pharmaceutically acceptable carriers, and, optionally, other therapeutic and/or prophylactic ingredients must be “acceptable” in the sense of being compatible with the other ingredients of the formulation and not be harmful to the recipient thereof. Suitable pharmaceutically acceptable carrier solutions include water, saline, isotonic saline, phosphate buffered saline, Ringer's lactate, and the like. The compositions of the present disclosure can be administered to animals, preferably to mammals, and in particular to humans as therapeutics per se, as mixtures with one another or in the form of pharmaceutical preparations, and which as active constituent contains an effective dose of the active agent, in addition to customary pharmaceutically innocuous excipients and additives.

The method can further include analyzing the subject using electrocardiogram. The methods can further include determining heart hemodynamics.

Suitable methods for administration of formulations of the present disclosure are by parenteral (e.g., intravenous (IV), intramuscular (IM), subcutaneous (SC), or intraperitoneal (IP)) routes and the formulations administered ordinarily include effective amounts of product in combination with acceptable diluents, carriers and/or adjuvants. Standard diluents such as human serum albumin are contemplated for pharmaceutical compositions of the disclosure, as are standard carriers as described herein. The composition can be administered over the course of days, weeks, months, and years.

Formulations for parenteral administration (e.g. by injection, for example bolus injection or continuous infusion) can be presented in unit dose form in ampoules, pre-filled syringes, small volume infusion or in multi-dose containers with and without an added preservative. The formulations can take such forms as suspensions, solutions, or emulsions in oily or aqueous vehicles, and may contain formulation agents such as suspending, stabilizing and/or dispersing agents.

As used herein, an “effective amount”, a “therapeutically effective amount”, a “prophylactically effective amount” and a “diagnostically effective amount” is the amount of the therapy of the present disclosure needed to elicit the desired biological response following administration. Suitable dosage for use in the methods of the present disclosure will depend upon a number of factors including, for example, age and weight of an individual, severity of the cardiomyopathy, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

As used herein, “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders associated with administration of the therapy.

### EXAMPLES

Analysis of the clinical literature identified that patients with deletion mutations in the region of R16 to R19 of dystrophin often display early onset and/or more severe heart disease. Two cardiac specific dystrophin transgenic mice were generated on the background of dystrophin-null mdx mice, the most commonly used mouse model for DMD. In one strain, a ΔH2-R19 micro-dystrophin was expressed in the heart. In the other strain, a ΔH2-R15 micro-dystrophin was expressed in the heart. The only difference between the ΔH2-R19 micro-dystrophin and the ΔH2-R15 micro-dystrophin was that ΔH2-R15 micro-dystrophin includes R16-19 but ΔH2-R19 micro-dystrophin does not.

On cardiac function analysis (ECG and cardiac catheter), the ΔH2-R15 micro-dystrophin offered better cardiac protection. Specifically, cardiac expression of the ΔH2-R15 mini-dystrophin normalized all aberrant ECG changes while cardiac expression of ΔH2-R19 mini-dystrophin did not. On cardiac catheter assay, both mini-dystrophins improved cardiac hemodynamics but only ΔH2-R15 mini-dystrophin corrected the end-diastolic volume, an important indication of diastolic heart function. Biochemistry study indicated that ΔH2-R15 mini-dystrophin was much more effective than ΔH2-R19 mini-dystrophin in recruiting cavin-1, a cardiac specific dystrophin binding protein. (see, Wasala N B, Shin J-H, Lai Y, Yue Y, Duan D. Cardiac specific expression of ΔH2-R15 mini-dystrophin normalized all ECG abnormalities and the end-diastolic volume in a 23-m-old mouse model of Duchenne dilated cardiomyopathy. Human Gene Therapy 29(7):737-748, 2018). Notably, dystrophin-bound cavin-1 is completely lost in the heart of mdx mice. Further, cavin-1 deficiency has been shown to contribute to muscular dystrophy and cardiomyopathy. The results of expressing ΔH2-R15 mini-dystrophin and ΔH2-R19 mini-dystrophin indicated that R16-19 can offer cardiac protection through enhanced recruitment of cavin-1. Inclusion of R16-19in mini-dystrophin or micro-dystrophin gene therapy vectors may lead to better heart protection.

To further test the therapeutic potential of R16-19 domain, a novel micro-dystrophin gene carrying R16-19 was engineered. Arrhythmia is a major cardiac concern in DMD patients. Loss of the Na current in Purkinje fibers underlies ventricular conduction defects and concomitant arrhythmias in the dystrophic heart. To test whether R16-19 μDys can rescue in Na current loss in dystrophic cardiac Purkinje fibers, AAV9.R16-19 μDys was injected into 8-week-old mdx-Cx40 eGFP mice. In this mdx strain, Purkinje fibers were genetically labelled with eGFP for unambiguous identification of the Purkinje fibers for electrophysiological studies. At 12 weeks post-injection, Na current was examined in wild type mice, mdx mice and R16-19 μDys treated mice. Results demonstrated that the Na current density was restored to the wild-type level in R16-19 μDys treated mice. Impaired Na channel inactivation was also rescued by R16-19 μDys therapy. By restoring wild-type Na current properties in dystrophic Purkinje fibers, the molecular underpinning of impaired ventricular conduction in the dystrophic heart was corrected. Our results suggest that R16-19 μDys can prevent and/or treat fatal arrhythmias in patients.

Six 8-week-old male mdx (C57BL/10ScSn-Dmdmdx/J)-Cx40_{eGFP/+} mice received a single tail vein injection of AAV9 μDys vector (6×10¹² viral genome particles/mouse). μDys contained the N-terminal and cysteine-rich domains, hinges 1 and 4, and spectrin-like repeats 16 to 19 of human dystrophin (FIG. 1A). R16-19 is a region implicated in heart protection. 12 weeks post-injection, mice were anesthetized using isoflurane (2%, inhalation) and euthanized by cervical dislocation. Thereafter, hearts were removed, and single Purkinje fibers were isolated from ventricular tissue according to our published protocol, whereby the Cx40_{eGFP/+} background allowed for unambiguous identification of Purkinje fibers for electrophysiological studies. Sodium currents of isolated Purkinje fibers were recorded with the whole-cell patch-clamp technique, and compared with sodium currents of Purkinje fibers isolated from age- and sex-matched untreated mdx-Cx40_{eGFP/+} and wild-type (C57BL/10ScSnJ)-Cx40_{eGFP/+} mice.

Twelve weeks after AAV μDys vector application to mdx-Cx40_{eGFP/+} mice, robust μDys expression was observed in the heart (FIGS. 1B-1E). Quantitative analyses indicated that nearly 100% of the cardiomyocytes expressed μDys (FIG. 1D). Purkinje fiber identity of eGFP-positive cells was confirmed in separate control experiments by detection of significant T-type Ca current (FIG. 1F, details given in legend). Whereas Purkinje fibers isolated from hearts of untreated mdx mice showed abnormally reduced Na currents, the Na current density in Purkinje fibers from AAV μDys vector-treated mdx mice was restored to the wildtype level (FIGS. 1G and 1H). Impaired Na channel inactivation, represented by a moderately slowed current decay in mdx compared to wild-type fibers, was also rescued by μDys therapy (FIG. 1I). Na channel activity in the Purkinje fiber membrane is a major determinant of ventricular conduction velocity. Thus, by restoring wild-type Na current properties in dystrophic Purkinje fibers, the molecular underpinning of impaired ventricular conduction and concomitant arrhythmias were corrected in the dystrophic heart.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

1. A composition for treating Duchenne muscular dystrophy in a subject having or suspected of having Duchenne muscular dystrophy comprising a microdystrophin comprising a dystrophin N-terminal domain, a dystrophin repeat 16 (R16) domain, a dystrophin repeat 17 (R17) domain, a dystrophin repeat 18 (R18) domain, a dystrophin repeat 19 (R19) domain, and a dystrophin cysteine rich (CR) domain.

2. The composition of claim 1, wherein the microdystrophin further comprises a dystrophin hinge 1 domain, a dystrophin hinge 4 domain, and combinations thereof.

3. (canceled)

4. The composition of any one of claims 1 or 2, wherein the composition comprises a nucleic acid encoding the microdystrophin.

5.-7. (canceled)

8. The composition of claim 4, wherein the nucleic acid encoding the microdystrophin further encodes a cardiac tissue-specific promoter.

9. A vector comprising a nucleic acid encoding a microdystrophin comprising a dystrophin N-terminal domain, a dystrophin R16 domain, a dystrophin R17 domain, a dystrophin R18 domain, a dystrophin R19 domain, and a dystrophin CR domain.

10. The vector of claim 9, wherein the nucleic acid encoding the microdystrophin further encodes a dystrophin hinge 1 domain, a dystrophin hinge 4 domain, and combinations thereof.

11. (canceled)

12. The vector of any one of claims 9 or 10, wherein the vector is selected from a viral vector and a non-viral vector.

13. The vector of claim 12, wherein the viral vector is an adeno-associated viral vector.

14. (canceled)

15. The vector of any one of claim 9, 10, 12, or 13, wherein the nucleic acid encoding the microdystrophin further encodes a cardiac tissue-specific promoter.

16. (canceled)

17. A method of treating Duchenne muscular dystrophy in a subject having or suspected of having Duchenne muscular dystrophy, the method comprising, administering to the subject a composition comprising a microdystrophin comprising a dystrophin N-terminal domain, a dystrophin R16 domain, a dystrophin R17 domain, a dystrophin R18 domain, a dystrophin R19 domain, and a dystrophin CR domain.

18. The method of claim 17, wherein the microdystrophin further comprises a dystrophin hinge 1 domain, a dystrophin hinge 4 domain, and combinations thereof.

19. (canceled)

20. The method of any one of claims 17 or 18, wherein the composition comprises a nucleic acid encoding the microdystrophin.

21. The method of claim 20, wherein the nucleic acid is selected from a viral vector and a non-viral vector.

22. The method of claim 21, wherein the viral vector is an adeno-associated viral vector.

23. (canceled)

24. The method of any one of claims 20 or 22, wherein the nucleic acid further encodes a cardiac-specific promoter.

25. (canceled)

26. A method of treating cardiac arrhythmia in a subject having or suspected of having Duchenne muscular dystrophy, the method comprising, administering to the subject a composition comprising a micro-dystrophin comprising a dystrophin N-terminal domain, a dystrophin R16 domain, a dystrophin R17 domain, a dystrophin R18 domain, a dystrophin R19 domain, and a dystrophin CR domain.

27. The method of claim 26, wherein the microdystrophin further comprises a dystrophin hinge 1 domain, a dystrophin hinge 4 domain, and combinations thereof.

28. (canceled)

29. The method of any one of claim 26 or 27, wherein the composition comprises a nucleic acid encoding the microdystrophin.

30. The method of claim 29, wherein the nucleic acid is selected from a viral vector and a non-viral vector.

31. (canceled)

32. (canceled)

33. The method of claim 29, wherein the nucleic acid further encodes a cardiac-specific promoter.

34.-38. (canceled)
